(19) United States
(12) Patent Application Publication
(10) Pub. No.: US 2023/0245779 A1
(43) Pub. Date: Aug. 3, 2023

(54) SYSTEM AND METHOD FOR PERI-ANAESTHETIC RISK EVALUATION

(71) Applicant: INTELLIGENCE ANESTHESIA, Paris (FR)

(72) Inventor: Yassine MOUSSALI, Paris (FR)

(73) Assignee: INTELLIGENCE ANESTHESIA, Paris (FR)

(21) Appl. No.: 18/002,709

(22) PCT Filed: Jun. 25, 2021
(86) PCT No.: PCT/EP2021/067475

§ 371 Date: Dec. 21, 2022

(30) Foreign Application Priority Data
Jun. 26, 2020 (EP) 20305712.0

(51) Int. Cl. G16H 50/30; G16H 10/20; G16H 10/60; G16H 20/40

(52) U.S. Cl. CPC G16H 50/30; G16H 10/20; G16H 10/60; G16H 20/40

(57) ABSTRACT

A peri-anesthesia risk assessment system configured to: receive patient data including the patient's answers to at least one patient questionnaire, data collected from at least one sensor and data from a patient data repository; from the received patient data, evaluate at least two index risks; calculate a global risk level based on the at least two index risks; select at least one recommendation from a library of recommendations, the at least one recommendation being selected by a perianesthetic assessment algorithm based on the at least two index risks and on the global risk level; receive as input data including information resulting from the user executing the at least one recommendation; modify the index risks based on the information resulting from the user executing the at least one recommendation; modify the global risk level based on the modified index risks.

### FIELD OF INVENTION

The present invention relates to the field of risk assessment in anesthesia. More precisely, the present invention relates to a system and a method for assessment of a peri-anesthetic risk of a patient, based on a set of patient data.

### BACKGROUND OF INVENTION

Risk assessment in anesthesia procedures are fundamental to reduce morbimortality associated with medical procedures conducted under anesthesia. The actual risk assessment methods are based on simple scoring systems, which do not have sufficient accuracy hence are inadequate for predictive and personalized care.

In the context of anesthesia, the American Society of Anesthesiologists (ASA) has developed a classification system to evaluate the anesthetic risk of a patient. The ASA classification is a categorization of a patient's physiological status in one among 6 categories, therefore this classification system alone does not provide an exhaustive prediction of the peri-operative risk of a patient.

### SUMMARY

The present invention relates to a computer-implemented method for assessing risks of a patient undergoing an anesthesia procedure, the method comprising the following steps:
receiving data relating to the patient health status, the received data comprising:
   the patient's answers a questionnaire relating to at least a respiratory status and a cardiac status of the patient;
   data collected from at least one sensor configured to sense physiological signals representative of the respiratory status and the cardiac status of the patient; and
   data from a repository comprising an anesthetic information and management record (AIMR) and/or an electronic medical record (EMR) of the patient;
labelling each of the received patient data according to a set of tags comprising a “respiratory” tag and a “cardiac” tag;
from the received patient data, calculating a number N of index risks, the index risks comprising at least one respiratory risk calculated based on the patient data labeled as “respiratory” and at least one cardiac risk calculated based on the patient data labeled as “cardiac”;
calculating a global risk level based on the index risks;
outputting the global risk level so as to provide the risk of the patient undergoing an anesthesia procedure;
   wherein the global risk level is calculated via the following equation:

Σᵢ₌₁^{N}kᵢ*Rᵢ

N being the number of calculated index risks (31), k being a weighting factor, R being a numerical value associated with each calculated index risk.

The respiratory status and the cardiac status of a patient have a significant impact on an anesthesia procedure, even in non-respiratory and non-cardiac surgeries.

Advantageously, by calculating a global risk level based on at least two index risks it is possible to provide a risk assessment which takes into account the overall health of the patient undergoing anesthesia and all the risks which may possibly occur before, during and after anesthesia.

This is not possible with conventional scoring systems, because each of them focuses on a narrow aspect of the patient health. Therefore, they may over- or underestimate the patient's risks.

Moreover, the global risk level allows to compare different patients.

In one embodiment, the method further comprises:
selecting at least one recommendation from a library of recommendations, the at least one recommendation being selected based at least on the global risk level;
receiving as input data relating to the health status of the patient measured after execution of the at least one recommendation;
modifying each of the N index risks based on the data relating to the health status of the patient measured after execution of the at least one recommendation;
calculating an updated global risk level based on the modified index risks.

In the modification step, each of the index risks may be multiplied by a predetermined correction factor, depending on the health status of the patient measured after execution of the at least one recommendation.

By selecting a recommendation on the basis of an index risk, it is possible to select the recommendation which minimizes said index risk.

By selecting a recommendation on the basis of the global risk level, it is possible to select the recommendation which minimizes morbimortality.

Contrarily to risk assessment methods in which an anesthetic recommendation is generated based on pre-anesthetic data, such as laboratory test results or pre-existing medical conditions, the present method ensures that the risk assessment is continuously improved based on information resulting from the user executing said recommendation. This information may change the anesthetic pathway of the patient. Therefore, the present method provides an improved risk assessment and patient care strategy that is safe and automatized.

Moreover, this method allows to automatically modify the calculated risks based on the information resulting from a user executing the recommendation.

In one embodiment, the method further comprises:
periodically, receiving data from a medical database comprising patient data and respective outcomes;
calculating at least one correlation between the received patient data and the respective outcomes;
modifying the inputs of the equation based on the at least one correlation;
optionally, outputting the modified first algorithm.

Advantageously, this embodiment allows to modify the inputs of the global risk level equation based on the calculated correlation. Therefore, it allows to improve the perianesthetic assessment algorithm based on evidence-based medicine.

Moreover, the modified algorithm may be outputted so as to receive a user validation of the modification.

More precisely, by “modifying the inputs of the global risk level equation” it is meant: modifying the number of calculated index risks, the weighting factor, and/or the numerical value associated with each calculated index risk.

In one embodiment, the method further comprises:
periodically, receiving medical guidelines from at least one medical guideline database, each medical guideline being received at a respective time, the medical guidelines being stored in a database and being associated with a label;
comparing each received medical guideline with the respective medical guideline associated with the same label and being received at a preceding acquisition time;
modifying the inputs of the equation based on the result of the comparison;
optionally, outputting the modified first algorithm.

Advantageously, this embodiment allows to modify the inputs of the global risk level equation in response to a change in the medical guidelines, so as to ensure that the selection of the recommendation is compliant with evidence-based medicine.

The present invention also relates to a computer program product for assessing the risks of a patient undergoing an anesthesia procedure, the computer program product comprising instructions which, when the program is executed by a computer, cause the computer to carry out the steps of the method according to any of the embodiments as described hereabove.

The present invention also relates to a computer-readable storage medium comprising instructions which, when the program is executed by a computer, cause the computer to carry out the steps of the method according to any of the embodiments as described hereabove.

The present invention also relates to a system for assessing risks of a patient undergoing an anesthesia procedure, the system comprising:
an input configured to receive data relating to the patient health status, the received data comprising:
   the patient's answers to at least one questionnaire relating to at least a respiratory status and a cardiac status of the patient;
   data collected from at least one sensor configured to sense physiological signals representative of the respiratory status and the cardiac status of the patient; and
   data from a patient data repository;
a memory configured to store the patient data;
a processing unit configured to:
   label each of the received data according to a set of tags comprising a “respiratory” tag and a “cardiac” tag;
   based on the received data, calculate a number N of index risks, the index risks comprising at least one respiratory risk calculated based on the patient data labeled as “respiratory” and at least one cardiac risk calculated based on the patient data labeled as “cardiac”;
   calculate a global risk level based on the index risks; and
an output for outputting the global risk level so as to provide the risk of the patient undergoing an anesthesia procedure,
   wherein the global risk level is calculated via the following equation:

Σᵢ₌₁^{N}kᵢ*Rᵢ,

N being the number of calculated index risks, k being a weighting factor, and R being a numerical value associated with each calculated index risk.

The at least one sensor mentioned hereinabove may be for instance: an optical sensor, a pressure sensor; a force sensor; a thermal sensor.

The data collected from the at least one sensor may comprise physiological data and identity data.

In one embodiment, the memory of the system is further configured to store a library of recommendations and the processing unit is further configured to:
select at least one recommendation from the library of recommendations based at least on the global risk level;
receive as input data relating to the health status of the patient measured after execution of the at least one recommendation;
modify each of the N index risks based on the data relating to the health status of the patient measured after execution of the at least one recommendation;
calculating an updated global risk level based on the modified index risks.

Advantageously, the system allows to automatize the patient care by automatically modifying the risk evaluation, based on the information resulting from the user executing the at least one recommendation.

In one embodiment, the system further comprises means for modifying the at least one recommendation selected by the processing unit.

In one embodiment, the memory is further configured to store a reference medical database comprising patient data and patient outcomes collected from a plurality of patients and wherein the processing unit is further configured to:
periodically, receiving data from a medical database comprising patient data and corresponding patient outcomes;
calculate at least one correlation between the patient data and patient outcomes;
modifying the inputs of the equation based on the at least one correlation.

Advantageously, the periodic analysis of the data in the reference medical database allows to ensure the relevance of the recommendations selected by the perianesthetic risk assessment algorithm. Thanks to the periodic monitoring of the relevance of the recommendation, and to the modification of the perianesthetic assessment algorithm based on the data analysis, the morbimortality during and after a medical procedure under anesthesia is minimized, thus providing a safe anesthetic assessment and care strategy.

This embodiment also allows to increase the medical knowledge about peri-anesthetic risks by discovering novel correlations and hence, novel index risks.

In one embodiment, the processing unit is further configured to:
periodically, receiving medical guidelines from at least one medical guideline database, each medical guideline being received at a respective time;
comparing each received medical guideline with the respective medical guideline being received at a preceding time;
modifying inputs of the equation based on the result of the comparison.

In order to compare the received medical guidelines with the respective guidelines received at a preceding time, the processing unit may be configured to label the received medical guidelines with a label.

In this case, each received medical guideline may be compared with one or more medical guidelines associated with the same label and being received at a preceding time.

Based on the result of the comparison step, the processing unit may be configured to automatically modify the inputs of the equation. The modified equation may be outputted by the output of the system, so as to receive a user validation, for instance via a user interface.

Advantageously, this embodiment allows to modify or suggest a modification of the perianesthetic risk assessment algorithm based on evidence-based medicine, thus improving the quality of the anesthetic assessment and care strategy.

This embodiment also allows to modify the recommendation selection based on changes in the local, national or international guidelines, thereby ensuring that the anesthetic assessment and care strategy is up-to-date.

In one embodiment, the processing unit is further configured to execute a machine learning algorithm configured to:
predict a patient outcome based on the patient data;
receive as input a measured patient outcome;
compare the predicted patient outcome with the received patient outcome to identify a discrepancy;
in case of discrepancy, anonymize and store the measured patient outcome and the patient data in the training dataset of the machine learning algorithm and/or in a reference medical database.

Preferably, the processing unit is configured to execute a machine learning algorithm configured to implement the aforementioned steps.

This embodiment allows to use discrepancies to optimize future predictions.

In one embodiment, the patient outcome and the measured outcomes are selected from a group comprising future admissions or discharges; pre-anesthetic outcomes; early or late post-anesthetic outcomes.

For instance, the predicted outcome and the measured outcome may be the in-hospital mortality.

In one embodiment, the output of the system is further configured to:
output a first pre-operative anesthesia evaluation comprising the index risk, the global risk level and the at least one recommendation;
output a second pre-operative anesthesia evaluation comprising the selected at least one recommendation; information resulting from the user executing the at least one recommendation; the modified index risks and the updated global risk level.

In one embodiment, the N index risks are selected from a group consisting of: respiratory risk, neurological risk, kidney failure risk, immunologic risk, chronic pain risk, vascular risk, hepatic risk, cardiac risk, airway management risk, nausea and vomiting risk, thromboembolic risk, hemorrhagic risk, allergy risk, anemia risk infection risk, delirium risk.

### DEFINITIONS

In the present invention, the following terms have the following meanings:
“Peri-anesthesia” refers to all the phases preceding, and/or during and/or following an anesthesia procedure.
“Semi-automated” refers to a method, wherein at least one step is computer-implemented and at least one step is manual.

### BRIEF DESCRIPTION OF THE DRAWINGS

The following detailed description will be better understood when read in conjunction with the drawings. For the purpose of illustrating, the system 1 is shown in the preferred embodiments. It should be understood, however that the application is not limited to the precise arrangements, structures, features, embodiments, and aspect shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

Moreover, it will be appreciated by those skilled in the art that any flow charts, flow diagrams, and the like represent various processes which may be substantially represented in computer readable media and so executed by a computer or processor, whether or not such computer or processor is explicitly shown.

Features and advantages of the invention will become apparent from the following description of embodiments of a system, this description being given merely by way of example and with reference to the appended drawings in which:

FIG. 1 is a block diagram representing schematically a particular mode of a system 1 compliant with the present disclosure.

FIG. 2 is a block diagram representing one non-limiting example of the steps of a method according to the invention. In this example, four index risks 31 are evaluated. For intelligibility purpose, in FIG. 2 the steps of the method are illustrated for one of the four index risks 31, notably for the respiratory risk.

FIG. 3 is a block diagram representing one non-limiting example of the method of the invention. The steps therein illustrated may be implemented by the processing unit 3 of the system 1 depicted in FIG. 1, for instance by executing a first rule-based algorithm for global risk level assessment, and a second machine learning algorithm for modifying the first algorithm.

FIG. 4 is a diagram representing one example of a second algorithm which may be executed by the processing unit 3 of the peri-anesthesia risk assessment system 1 according to the present invention. In this example, the patient outcome is the blood loss, and a discrepancy exists between the predicted patient outcome and the measured patient outcome (i.e., the delta between the predicted blood loss and the measured blood volume loss). In the illustrated example, the second algorithm is a deep learning algorithm, and the discrepancy is retro-propagated in the previous layers of the deep learning algorithm, thereby allowing to improve future predictions.

FIG. 5 is a flowchart representing the main steps of a method for assessing a global risk level 32 according to the invention.

FIG. 6 is a flowchart representing one example of a method for assessing a global risk level 32 selecting a recommendation according to the invention.

FIG. 7 is a graph representing the in-hospital mortality (vertical axis) with respect to a global risk level 32 (horizontal axis) calculated with the method of FIG. 5.

While various embodiments have been described and illustrated, the detailed description is not to be construed as being limited hereto. Various modifications can be made to the embodiments by those skilled in the art without departing from the true spirit and scope of the disclosure as defined by the claims.

### DETAILED DESCRIPTION

The present description illustrates the principles of the present disclosure. It will thus be appreciated that those skilled in the art will be able to devise various arrangements that, although not explicitly described or shown herein, embody the principles of the disclosure and are included within its scope.

All examples and conditional language recited herein are intended for educational purposes to aid the reader in understanding the principles of the disclosure and the concepts contributed by the inventor to furthering the art, and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

The present invention relates to a system 1 and a method to assess risks of a patient undergoing an anesthesia procedure. In particular, the present invention allows to assess peri-anesthetic risks, i.e., risks encompassing all the events related to an anesthetic procedure.

Though the presently described system 1 is versatile and provided with several functions that can be carried out alternatively or in any cumulative way, other implementations within the scope of the present disclosure include systems having only parts of the present functionalities.

Each of the systems 1 is advantageously an apparatus, or a physical part of an apparatus, designed, configured and/or adapted for performing the mentioned functions and produce the mentioned effects or results. In alternative implementations, any of the system 1 is embodied as a set of apparatus or physical parts of apparatus, whether grouped in a same machine or in different, possibly remote, machines. The system 1 may e.g. have functions distributed over a cloud infrastructure and be available to users as a cloud-based service, or have remote functions accessible through an API.

Moreover, in what follows, the modules are to be understood as functional entities rather than material, physically distinct, components. They can consequently be embodied either as grouped together in a same tangible and concrete component, or distributed into several such components. Also, each of those modules is possibly itself shared between at least two physical components. In addition, the modules are implemented in hardware, software, firmware, or any mixed form thereof as well. They are preferably embodied within at least one processor of the system 1.

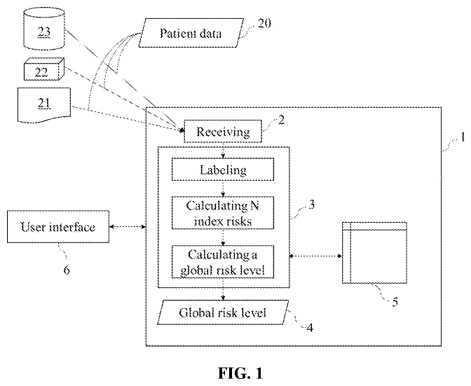

One exemplary system 1 according to the present disclosure is shown in FIG. 1. This system comprises an input 2 (also referred to as “acquisition module”), a processing unit 3, an output 4 (also referred to as “output module”), and a memory 5.

Moreover, the system 1 depicted in FIG. 1 is interacting with a user interface 6, via which information can be entered and retrieved by a user. The user interface 6 includes any means appropriate for entering or retrieving data, information or instructions, notably visual, tactile and/or audio capacities that can encompass any or several of the following means as well known by a person skilled in the art: a screen, a keyboard, a trackball, a touchpad, a touchscreen, a loudspeaker, a voice recognition system.

As shown in FIG. 1, the input 2 of the system 1 is configured to receive patient data 20 relating to the patient health status.

The received patient data 20 may comprise the patient's answers to at least one questionnaire 21, data collected from at least one sensor 22, and data from a patient data repository 23.

In the present invention, the at least one questionnaire 21 may comprise:
a first list of standardized questions;
a second list of personalized questions based on the patient's answers to the standardized questions;
a third list of personalized questions based on the patient data collected from the at least one sensor 22.

The questionnaire may comprise questions such as for example: «Do you snore loudly?», «Do you often feel tired, fatigued or sleepy during the daytime?»; «Has anyone observed you stop breathing or choking/gasping during your sleep?»; «Are you being treated or have you been treated for high blood pressure?»; «Is your body mass index superior to 35 kg/m²?»; «Are you older than 50 years old?».

The questionnaire may further comprise questions from scoring systems such as ASA, STOP-BANG, Lee, MET, and the like.

The second and third lists of questions may be generated by an algorithm executed by the processing unit 3 of the system 1.

The questions of the questionnaire 21 are configured to assess the health status of the patient and, advantageously, at least the respiratory and the cardiac status of the patient. Accordingly, the answers to the questionnaire 21 comprise at least the respiratory status and the cardiac status of the patient.

By “cardiac status” it is meant any information relating to the cardiac functional capacity to meet the metabolic demands of the patient.

By “respiratory status” it is meant any information relating to the respiratory functional capacity to meet oxygen demands and carbon dioxide extraction of the patient, i.e., relating to the capacity to exchange of carbon dioxide and oxygen at the alveolar level.

The cardiac and respiratory statuses have a large impact on a surgery outcome, even in non-cardiac and non-respiratory surgery. By receiving these two statuses it is possible to assess at least the cardiac and respiratory risks 31 of the patient undergoing the anesthesia procedure.

Optionally, the questions of the questionnaire 21 may be configured to further assess other health statuses such as: nutritional status, psychological status, renal status, neurological status, renal status, hepatic status, immunologic status.

As aforementioned, the input 2 is further configured to receive patient data 20 collected from at least one sensor 22. Said sensor 22 is configured to sense physiological signals representative of the health status of the patient. Preferably, the sensor 22 is configured to sense physiological signals relating at least to the respiratory status and the cardiac status.

In this case, a single sensor 22 may sense a physiological signal relating both to the respiratory status and the cardiac status. One example of physiological signal relating both to the respiratory status and the cardiac status is the cardiac output, which it is the amount of blood pumped by the heart in one minute, but it is also related to the respiratory status (the rate and depth of respiration affect the venous return and, therefore, the cardiac output).

Alternatively, the input 2 may receive the data collected by two distinct sensors 22, each of them being configured to sense the respiratory status or the cardiac status.

The physiological signal relating to the cardiac status may comprise: blood pressure, heart rate, heart rhythm, cardiac output, stroke volume, cardiac preload, vascular resistance, cardiac afterload, myocardial contractility and the like.

The physiological signal relating to the respiratory status may comprise: recording of the respiratory auscultation, oxygen saturation, cardiac output, rate of breathing, rhythm of breathing (i.e., regularity of the respiration patterns and the pauses between each breath) and the like.

The at least one sensor 22 may be for instance: an optical sensor, a pressure sensor; a force sensor; a thermal sensor.

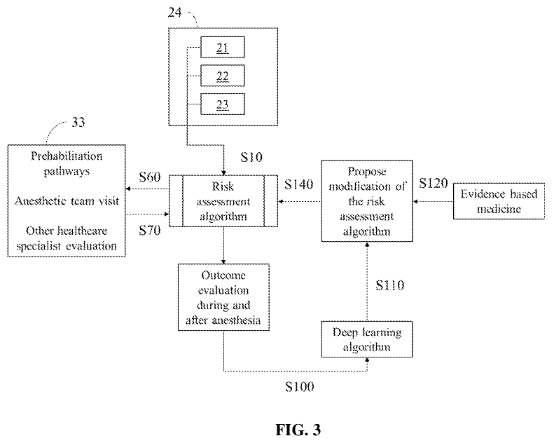

As shown in FIG. 3, the at least one sensor 22 may be a sensor of a medical device 24, such as for example a Sp0₂ sensor, an ECG Sensor, a PPG sensor or a sensor of a health-related smart device, such as for example a smartwatch, a fitness tracker wristband, a biopatch, a wearable device.

In this case, the sensor 22 records one or more measurements which are transferred to a transmitter of the medical device 24, said transmission may be done through a wire or wirelessly. The measurements are then transferred via communication network to the system 1.

In one embodiment, the at least one sensor 22 is a sensor of a mobile phone or smartphone, such as for example a camera sensor. This embodiment allows to provide to the system 1 identification data, such as a photograph of the patient or a photograph of a patient's identity document. In addition, the sensor 22 according to this embodiment may provide a photograph of a medical document in paper format.

As aforementioned, the input 2 is further configured to further receive patient data from a repository 23.

In a preferred embodiment, the patient data repository 23 is an anesthetic information and management record (AIMR) or an electronic medical record (EMR).

The patient data repository 23 may be stored in one or more local or remote database(s). The latter can take the form of storage resources available from any kind of appropriate storage means, which can be notably a RAM or an EEPROM (Electrically-Erasable Programmable Read-Only Memory) such as a Flash memory, possibly within an SSD (Solid-State Disk).

Accordingly, the patient data repository 23 may be a repository stored in the memory 5 of the system 1.

Alternatively, it may be a repository 23 stored in a database of a healthcare facility, such as for example an AIMR stored in an electronic medical record system of a medical facility.

The patient data repository 23 may a be a repository residing on a non-transitory storage media of a device 24 such as the patient's smartphone or mobile telephone. In this case, the device 24 comprises a transmitter configured to transmit the patient data in the repository 23 to the system 1. The transmission may be done through a wire or wirelessly.

The patient data 20 from the repository 23 may comprise a scan or a photograph or medical documents in a paper format.

The patient data 20 from the data repository 23 may comprise results of biological screening tests and analysis; medical prescription; genetic profiling; clinical data from patient autonomous auscultation; blood pressure; oxygen saturation and multi-spectrophotometric analysis; arterial pulse wave; electrocardiogram; psychomotricity test result; cardiac stress test result.

As aforementioned, the system 1 may comprise a memory 5. The memory 5 may be configured to store the received patient data 20.

Preferably, the memory 5 is a non-transitory, processor-readable memory 5. The processing unit 3 may read and execute instructions stored on said memory 5 in order to perform a risk assessment method.

The expression “processing unit” should not be construed to be restricted to hardware capable of executing software, and refers in a general way to a processing device, which can for example include a computer, a microprocessor, an integrated circuit, or a programmable logic device (PLD). The processor may also encompass one or more Graphics Processing Units (GPU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.

In the present invention, the processing unit 3 is configured to calculate a number N of index risks 31, based on the received patient data 20. For instance, the processing unit may be configured to execute a first algorithm for performing said calculation.

Preferably, the processing unit 3 is configured to execute a first algorithm, also called “perianesthetic assessment algorithm” for calculating the global risk level 32, and a second algorithm, also called “improvement algorithm”. The first algorithm is preferably a rule-based algorithm.

The processing unit 3 may further execute additional algorithms, for example to generate the second and the third list of questions of the patient questionnaire; and/or to predict patient outcomes.

For instance, the first algorithm may be configured to:
based on the patient data 20, evaluate at least two index risks 31;
evaluate a global risk level 32 (also referred to as “global anesthetic risk level”) based on the index risks 31.

As aforementioned, the system 1 may comprise a memory 5. The memory may be configured to store a library of recommendations 33. In this case, the first algorithm may be configured to:
select at least one recommendation 33 from the library of recommendations based on the index risks 31 and/or on the global anesthetic risk level 32.

The processing unit 3 may further be configured to modify the at least one recommendation 33. In this case, the first algorithm may be configured to:
receive as input data comprising information resulting from the user executing the at least one recommendation 33;
modify each index risk 31 based on the information resulting from the user executing the at least one recommendation 33;
modify the global anesthetic risk level 32 based on the modified at least one index risk.

The processing unit 3 may also be configured to classify, or label, the received data according to a set of classes, or tags. Preferably, said classes, or tags, comprise “respiratory” and “cardiac”.

Advantageously, this embodiment allows to calculate the index risks 31 having a major impact on an anesthesia procedure, namely a respiratory risk, which is calculated on the basis of the data 20 classified or labeled as “respiratory”, and a cardiac risk, which is calculated on the basis of the data 20 classified or labeled as “cardiac”.

In a variant, the first algorithm may be configured to calculate other index risks 31, alternatively or in combination with the respiratory and cardiac risks.

Preferably, the processing unit 3 is configured to evaluate at least two index risks 31.

The index risks 31 are preferably selected from a group comprising: respiratory risk, neurological risk, kidney failure risk, hepatic risk, cardiac risk, airway management or airway accessibility risk, nausea and vomiting risk, thromboembolic risk, hemorrhagic (or bleeding) risk, immunologic risk, chronic pain risk, vascular risk, allergy risk, infection risk, delirium risk.

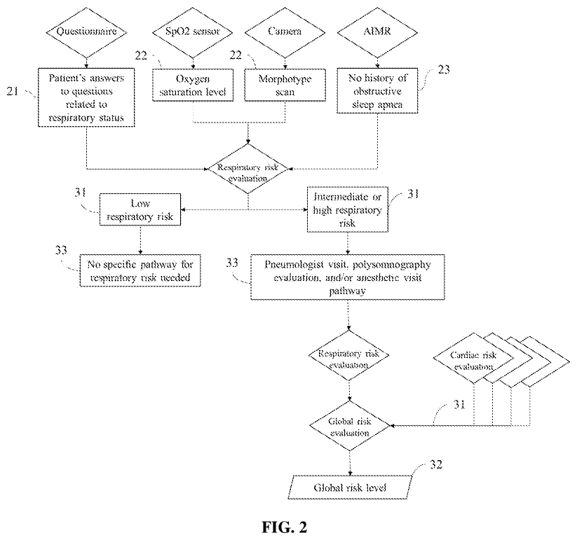

In the embodiment represented in FIG. 2, the system 1 evaluates four index risks 31. In this example, the four index risks 31 are calculated on the basis of patient data 20 comprising the patient's answers to at least one questionnaire 21; data collected from at least one sensor 22 and data from a patient data repository 23.

More precisely, in this example the data collected from at least one sensor 22 comprise oxygen saturation levels obtained by a SpO2 sensor and a morphotype scan obtained via a camera. The data from a patient data repository 23 may comprise data related to a history of obstructive sleep apnea that are stored in an anesthetic information and management record (AIMR).

In a preferred embodiment, the system of the present invention evaluates a number of index risks 31 comprised between 2 and 15.

In one embodiment, the index risk 31 is a categorical variable; such as for example: low risk, intermediate risk or high risk. One example of this embodiment is represented in FIG. 2.

In one embodiment, the index risk 31 is a continuous variable.

The processing unit 3 is configured to calculate the global risk level 32 based on each index risk 31.

By calculating the global risk level 32 based on at least two index risks 31, it is possible to provide an objective assessment (the global anesthetic risk level 32, or global risk level 32) of the overall health status and risks of a patient undergoing anesthesia.

Inversely, conventional risk scoring systems focus on a narrow aspect of the patient health. Accordingly, they provide a score which is indicative of a specific risk (for instance: indicative of preoperative physical fitness, or breathing disorders, or cardiac disorders), thereby neglecting other anesthesia-related risks.

More precisely, the processing unit 3 is configured to calculate the global risk level 32 via equation (e1):

Σᵢ₌₁^{N}kᵢ*Rᵢ (e1),

wherein N is the number of calculated index risks, which is equal or greater than 2, k is a weighting factor and R is a numerical value associated with the calculated index risk 31.

The processing unit may be configured to calculate each weighting factor k on the basis of the respective index risk 31 and the type of surgery. For instance, the weighting factor k for a renal failure risk would be higher for a kidney surgery and lower for a cardiac surgery.

Moreover, in some embodiments the input 2 of the system 1 may receive data related to the type of surgery from the repository 13. The type of surgery may be inputted by a user via the user interface 6.

In this case, the processing unit 3 may be configured to calculate the global risk level 32 via the equation (e2):

Σᵢ₌₁^{N}S*kᵢ*Rᵢ (e2),

wherein S is a constant relating to the type of surgery. The processing unit 3 may be configured to select the constant S on the basis of the type of surgery. For instance, S may be equal to 1 for a medium risk surgery, inferior to 1 for a low-risk surgery, and greater than 1 for a high-risk surgery.

If one or several index risks 31 are categorical variables, the processing unit 3 may be configured to convert them from categorical variable to a numerical value prior calculation of the global risk level 32.

Advantageously, the global risk level 32 thus obtained is correlated with mortality. Therefore, it allows to identify patients with a higher risk of morbi-mortality.

The global risk level 32 thus obtained is a numerical value, preferably a continuous variable.

Advantageously, the global risk level 32 allows to detect subtle changes in the overall risks of the patient, which may not affect the conventional scoring systems. Accordingly, it also allows to provide a fine patient assessment, and to identify subtle differences between two or more patients. This is not possible with coarse scoring systems such as the ASA classification.

Advantageously, the global risk level 32 ensures that a unique value is used to assess the risks of the patient, said global risk level 32 conveying information relating to the overall health of the patient undergoing anesthesia and to his/her anesthesia-related risks.

The processing unit 3 may further be configured to convert the global risk level 32 from numeric to categorical variable. In this case, the global risk level 32 and/or the respective categorical variable may be outputted via the output 4 of the system 1. For instance, by displaying the global risk level 32 on the user interface 6 of the system 1, it is possible to communicate patient's overall health to a healthcare provider.

By displaying the global risk level 32 on the user interface 6 of the system 1, it is possible to communicate patient's overall health to a healthcare provider.

The global risk level 32 thus obtained has several advantages.

First of all, it provides a “big picture” of the patient to the healthcare provider. This is not possible with the currently used scoring systems, such as the ASA classification. Indeed, these scoring systems focus on a specific risk, and each of them is evaluated independently from the other scoring systems. Therefore, they do not provide information about the patient as a whole.

Moreover, it is currently not possible to compare patients evaluated with different scoring system, and determine whom of them is at higher risk. For instance, it would not be possible to objectively compare two patients based on a Lee's score of the former and an ASA class of the latter.

Inversely, the global risk level 32 allows to compare two or more patients, even if the data 20 acquired for each patient are of different nature.

It should also be noted that the currently used scoring systems are subjected to bias due to the fact that they intrinsically focus on a narrow aspect of the patient's health, which leads to over- or underestimation of the patient's risk.

For instance, the ASA score is based on preoperative physical fitness, it is subjective, and it cannot take into account the type of surgery; the LEE score takes into account the type of surgery, but it is based on six predictors of cardiac complications and it cannot estimate risks other than the cardiac risk. Moreover, scoring system merely based on a cardiac risk, may be misleading in assessing the risk of patients experiencing pain, due to the influence of pain over blood pressure and pulse rate. Furthermore, the currently used scoring systems do not take into account the psychological status of the patient, thereby underestimating risks related to the psychological status, such as postoperative pain.

Overall, the currently used scoring system do not provide an objective and unique parameter relating to the global risks of the patients undergoing anesthesia. This information may only be speculated by physicians in a subjective manner Therefore, the conclusion about the patient's situation depends on the physician's experience.

As aforementioned, the processing unit 3 of the system 1 may also be configured to select a recommendation 33 on the basis of the global risk level 32.

As the global risk level 32 is correlated with mortality, by selecting the recommendation 33 on the basis of the global risk level 32, it is possible to select the recommendation 33 which minimizes the morbimortality risk.

Preferably, the processing unit 3 is capable of executing a first algorithm for selecting at least one recommendation 33 based on the global risk level 32, and at least one recommendation 33 based on at least one predetermined index risk 31. In this case, the former allows to minimize the mortality risk, whereas the latter allows to minimize the at least one predetermined index risk 31.

Examples of recommendations 33 selected based on the global risk level 32 comprise: “Do not perform surgery”, “Provide postoperative follow-up in ICU for at least 5 days”, “Transfer the patient to a primary facility”. An exemplary recommendation 33 which may be selected based on the cardiac risk 31 is: “higher doses of statin and beta-blocker prior to surgery”.

In the example of FIG. 2, a recommendation 33 comprising a pneumologist visit, a polysomnography test, and/or an anesthetic visit is selected in case the respiratory risk is intermediate or high.

The at least one recommendation 33 may comprise for instance:
a nutritional recommendation, such as for example a diet, or a fasting period;
an activity recommendation, such as a physical exercise;
an educational recommendation, such as the educational material provided in oral, written, visual or electronic format.

As shown in FIG. 3, the processing unit 3 may also be configured to receive data relating to the health status of the patient measured after execution of the at least one recommendation 33.

Advantageously, in this case the system 1 allows to automatize the patient care by improving the risk evaluation based on the information resulting from the user executing the at least one recommendation 33.

The information resulting from the user executing the at least one recommendation 33 may comprise for instance the patient weight; the patient's answer to a self-test comprising questions configured to assess the patient's understanding of educational material or to assess the patient's psychological status.

For instance, the at least one recommendation 33 may be an educational recommendation, comprising a video configured to inform the patient about regional anesthesia followed by a self-test configured to assess the patient's psychological status. Advantageously, this embodiment allows to modify the index risks 31 and the global risk level 32 and hence the recommendation 33, based on the patient's psychological status.

For instance, if the patient autonomously reports a panic attack related to the visualization of the video, or if the patient's answers to the self-test indicate a panic attack episode, the index risks 31 and the global risk level 32 may be modified accordingly. Because of the panic attack episode, the modified global risk level may be higher than the previously calculated global risk level 32. As a result, if a regional anesthesia was initially chosen based on the global risk level 32, a general anesthesia may replace the initial choice, based on the modified global risk level 32b.

The selected recommendation 33 may comprise a diet and an objective of weight. In this case, the data comprising information relating to the health status of the patient measured after execution of the recommendation 33 may be the patient weight. The first algorithm may receive as input the patient weight and it may be configured to modify each patient index risk 31 and the global risk level 32 based on such information. If the patient weight corresponds to the objective of weight the algorithm may modify the index risks 31 and/or the global risk level 32.

In one embodiment, the at least one recommendation 33 comprises a medical pathway and/or an anesthetic protocol. Said medical pathway may comprise one or more anesthesiologic consultations and non-anesthesiologic consultations. The medical pathway is an optimized medical pathway to properly evaluate the anesthetic risk of the patient. In this case, the user executing the at least one recommendation 33 is the anesthesiologist or another healthcare specialist, such as a radiologist, a pneumologist, a cardiologist. Moreover, the recommendation 33 may further comprises a consultation modality, selected based on a predetermined index risk 31 and/or on the global risk level 32. For instance, said modality may be an in-person modality or a remote modality.

As aforementioned, the at least one recommendation 33 may comprise an anesthetic protocol. In this case, by selecting the anesthetic protocol on the basis of the global risk level 32, it is possible to provide the protocol comprising the anesthesia procedure that minimize the risk of morbimortality related to the medical procedure performed under anesthesia.

The information resulting from the user executing the at least one recommendation 33 may comprise information that come to light during a preoperative consultation, such as for example the discovery of a severe aortic stenosis by a cardiologist, or the discovery of a hemophilia by a hematologist, or a severe sleep apnea syndrome by a pneumologist.

In this case, the information resulting from the user executing the at least one recommendation 33 may comprise per-anesthetic information, such as for example a clinical decision support for optimal antibio-prophylaxis or the cardiac output monitoring to use for one specific patient for a specific surgery, and/or post-anesthetic information such as for example the thrombo-embolic prevention needs, or the modality of blood test monitoring.

In one embodiment, the present invention further comprises means for transmitting the selected recommendation 33 to the patient and to the anesthetic staff.

The system 1 may further comprises means for modifying the at least one recommendation 33. For instance, the recommendation 33 may be modified by a healthcare provider. This embodiment allows to provide a semi-automated system 1 for supporting patient care and clinical decision.

As shown in FIG. 3, the processing unit 3 may be configured to execute a second algorithm for periodically modifying or suggest a modification of the first algorithm.

Advantageously, the second algorithm allows to increase the accuracy of the index risk 31 evaluation and the global anesthetic risk level 32 calculation over time. Therefore, it allows to improve the relevance of the recommendation 33.

The second algorithm may be a machine learning or deep learning algorithm.

Preferably, the second algorithm is a hybrid algorithm, i.e., it comprises a machine learning, or deep learning, algorithm and a set of rules.

In one embodiment, the second algorithm is configured to periodically, analyze data in a medical database to identify at least one correlation; and to modify or suggest a modification of the perianesthetic risk and assessment algorithm based on the result of the analysis.

More precisely, in this case the second algorithm may be configured to:
periodically, receive data from a medical database comprising patient data 20 and respective outcomes;
calculate at least one correlation between the received patient data 20 and the respective outcomes;
modifying the inputs of the equation e1 (or equation e2) based on the at least one correlation;
optionally, outputting the modified first algorithm.

Each patient data 20 may be linked in the medical database to a plurality of patient outcomes.

In this case, the second algorithm may calculate correlation between each patient data and the corresponding linked outcomes. The second algorithm may modify or suggest a modification of the first algorithm if at least one of said correlations is above a predefined threshold.

The medical database is preferably stored in the memory 5 of the system. Said database may comprise data 20 obtained from a healthcare facility.

In a preferred embodiment, said medical database is a dynamic database comprising data collected from a plurality of patients evaluated for an anesthetic risk by means of the first algorithm of the present invention.

In a preferred embodiment, said medical database is a dynamic database comprising data collected from a plurality of patients evaluated for an anesthetic risk by means of the first algorithm of the present invention.

In this case, the system 1 receives at least one measured outcome for each patient evaluated by the first algorithm. The measured outcome may be an outcome measured during or after anesthesia, such as for example early clinical outcomes such as walking distance capabilities or quantity of excreted urine during or after the surgery, biological outcomes such as the serum hemoglobin level or serum lactic acid level twenty-four hours after a surgery or late outcomes such as death or chronic pain syndrome.

The patient data 20, the patient risk indexes 31; the global anesthetic risk level 32; the at least one recommendation 33; the information related to the execution of the recommendation and the measured patient outcome may be stored in the medical database.

The data 20 are stored in the medical database so as to build a medical dataset. Preferably, the dataset comprise data collected from more than 100000 patients. Such data may comprise patient data 20 such as the answers to the questions of questionnaires 21, data collected from sensors 22 and data stored in patient data repositories 23; and patient outcomes. In this case, the second algorithm may process said data in order to calculate correlations between the patient data 20 and the patient outcomes.

Advantageously, this embodiment allows to increase the medical knowledge about peri-anesthetic risks by discovering novel correlations.

In one embodiment, the second algorithm is configured to periodically interrogate a medical guideline database about updated guidelines and modify or suggest a modification of the perianesthetic assessment algorithm based on the result of the interrogation.

More precisely, in this case the second algorithm is configured to:
periodically, receiving medical guidelines from at least one medical guideline database, each medical guideline being received at a respective time, the medical guidelines being stored in a database and being associated with a label;
comparing each received medical guideline with the respective medical guideline associated with the same label and being received at a preceding time;
modifying inputs of the equation e1, e2 based on the result of the comparison.

In this embodiment, the second algorithm is configured to modify or suggest a modification of the perianesthetic assessment algorithm based on evidence-based medicine. For instance, the modification may be based on the results of a prospective randomized controlled trial or a retrospective study.

Advantageously, this embodiment allows to identify novel index risks 31 related to anesthesia, such as a genetic risk, and automatically add the identified index risk 31 to the other index risks 31 introduced as input of the equation e1, e2 for global risk level 32 assessment.

In one embodiment, the updatable medical databases comprise anesthetic guidelines database, such as MEDLINE/PubMed; ASA standards and practice guidelines; eGuidelines; Guideline Central; National Institute for Clinical Excellence; National Library for Health (NLH) guidelines database.

In one embodiment, the second algorithm is configured to:
predict a patient outcome based on the patient data 20;
receive as input a patient outcome;
compare the predicted patient outcome with the received patient outcome to identify a discrepancy;
in case of discrepancy, anonymize and store the measured patient outcome and the corresponding patient data 20.

As aforementioned, the second algorithm may be a machine learning model. In this embodiment, if the discrepancy matches the rules in a set of plausibility rules, the patient data 20 and the received patient outcome may be added to the training set.

In one embodiment, the second algorithm hybrid algorithm which comprises a machine learning algorithm and a rule-based algorithm. In this embodiment, if the discrepancy matches all the rules in the set of plausibility rules, the rules of the predictive model may be modified. Further rules may be added to the predictive model to handle the clinical situation generating the discrepancy.

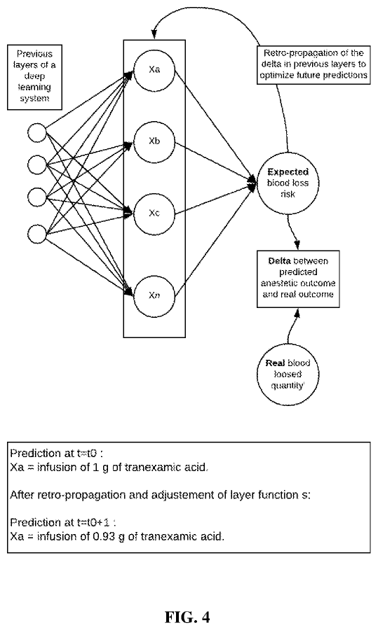

In one embodiment, the training of the machine learning model comprises calculating a delta related to the discrepancy and training hidden layers through a delta retro-propagation algorithm, as shown in FIG. 4.

In one embodiment, the validation of the machine learning model comprises: validating the model against a set of consistency rules and validating the model against a set of clinical reference cases.

In one embodiment, the processing unit 3 is further configured to store in the reference medical dataset: the index risk 31; the global risk level 32; the selected recommendation 33; the data comprising information resulting from the user executing the recommendation 33; the modified index risk; the modified global risk level 32b.

This embodiment allows to collect structured data and use them to improve the accuracy of future recommendations 33.

The discrepancy identification also allows identifying an unforeseen complication in the clinical outcomes or a non-adherence of the patient or a healthcare provider to the selected recommendation 33.

In one embodiment, the predicted outcomes and the received outcomes are selected from a group comprising future admissions or discharges; pre-anesthetic outcomes; early or late post-anesthetic outcomes.

One example of this embodiment is represented in FIG. 4. In this embodiment, the second algorithm predicts several variables Xa, Xb, Xc, . . . Xn and, based on the predicted variables, it predicts a patient outcome.

In the example illustrated in FIG. 4, the predicted variables are variables related to a blood loss risk, such as for example a tranexamic acid dosage, and the predicted patient outcome is a blood loss. The algorithm receives as input the measured blood volume lost; a discrepancy between the predicted blood loss and the received blood volume lost is identified, and the delta related to said discrepancy is retro-propagated in the previous layers of the second algorithm so as to improve future predictions.

In this particular example, the predicted variable Xa is the amount of tranexamic acid to be infused; the amount predicted by the second algorithm is 1 g and the amount predicted after delta retro-propagation is 0.93 g.

In one embodiment, the predicted outcome is selected from a group comprising: information resulting from the user executing the at least one recommendation; a complication, an early post-anesthetic event, a late post-anesthetic event, the need for post-anesthetic medical assessment, the modality of healthcare such as ambulatory or inpatient strategy, the post-anesthetic analgesic, and usual medication adjustment, improved anesthetic assessment for future anesthesia.

In one embodiment, the system comprises means for modifying the selected recommendation 33. In this embodiment, the processing unit 3 identifies a discrepancy between the selected recommendation 33 and the modified recommendation. Such discrepancy may be anonymized and stored in the medical dataset.

In one embodiment, the system further comprises an output 4, also referred to as “output module”.

The output 4 is for outputting the global risk level 32 so as to provide the risk of the patient undergoing an anesthesia procedure

The output 4 may further be configured to output a first pre-anesthesia evaluation comprising the global anesthetic risk level 32; and the selected recommendation 33.

In this case, the output 4 may further be configured to output a second pre-anesthesia evaluation comprising the selected at least one recommendation 33; the information resulting from the user executing said at least one recommendation 33; the modified global anesthetic risk level 32.

The present invention also relates to a computer-implemented method to assess the risks of a patient undergoing an anesthesia procedure.

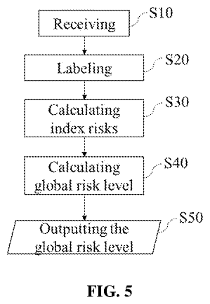

The main steps of the method are illustrated in FIG. 5.

The method comprises a step S10 of receiving patient data 20 comprising the patient's answers to at least one patient questionnaire 21, data collected from at least one sensor 22 and data from a patient data repository 23.

The method may further comprise a step S20 of labelling the received data according to a set of tags comprising a “respiratory” tag and a “cardiac” tag.

The method may further comprise a step S30 of evaluating at least two patient index risks 31 from the received patient data 20.

More precisely, step S30 comprises calculating a number N of index risks 31, N being equal or greater than 2. Preferably, the index risks 31 comprise at least one respiratory risk calculated based on the patient data labeled as “respiratory” and at least one cardiac risk calculated based on the patient data labeled as “cardiac”.

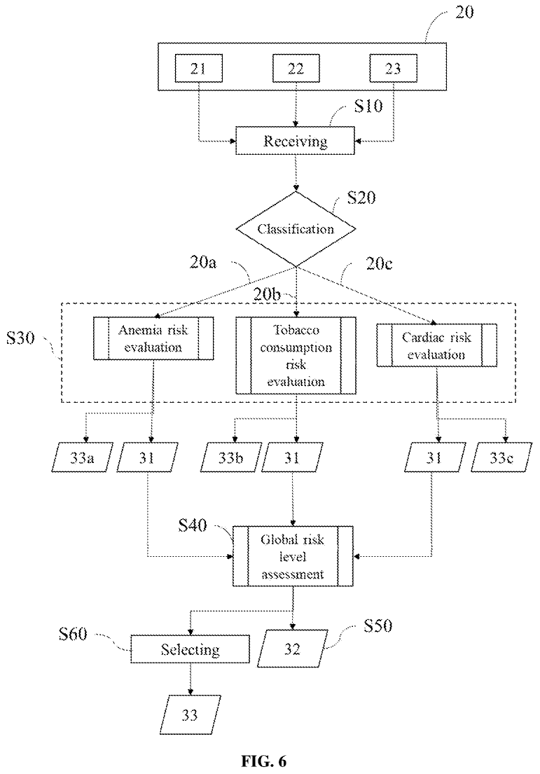

FIGS. 6 illustrates the main steps of a method compliant with the present disclosure, for assessing three risks.

In this case, the method may comprise identifying, in the received patient data 20, a first subset of data 20a relevant to an anemia risk, a second subset of data 20b relevant to a tobacco consumption, and a third subset of data 20c relevant to a cardiac risk.

The first subset of data 20a may comprise data such as for instance: type of surgery, iron pills assumption, anterior hemoglobin level, repetitive bleeding medical history.

The second subset of data 20b may comprise for instance a smoking history.

The third subset of data 20c may comprise for instance: cardiac surgery history, fitness level, cardiac arrhythmia history, cardiac electrocardiogram, cardiologist medical report, distance and elevation walked in a predetermined time, heart rate.

Some received data 20 may be associated to more than one set 20a, 20b, 20c. For instance, an anterior hemoglobin level may be associated both to the first and the second subsets of data 20a, 20b.

The method may further comprise a step S40 of calculating a global risk level 32 based on the at least two index risks 31.

The method may further comprise a step S50 of outputting the global risk level 32 so as to provide the risk of the patient undergoing an anesthesia procedure.

The global risk level 32 is calculated via equation e1:

Σᵢ₌₁^{N}kᵢ*Rᵢ (e1),

wherein N is the number of calculated index risks 31, k is a weighting factor, and R is a numerical value associated with each calculated index risk.

The method may further comprise a step S60 of selecting at least one recommendation 33 from a library of recommendations based on the index risks 31 and/or on the global risk level 32.

In the example of FIG. 6, three recommendations 33a, 33b, 33c are selected S60 on the basis of a respective index risk 31 and one recommendation 33 is selected S60 on the basis of the global risk level 32. For instance, the recommendation 33a selected based on the anemia risk may be an intravenous iron injection prior to surgery; the recommendation 33b selected based on the tobacco-consumption risk may be a nicotine substitution patch; the recommendation 33c selected based on the cardiac risk may be beta-selective sympathomimetic drug.

The method may further comprise a step S70 of receiving as input data comprising information resulting from the user executing the at least one recommendation.

More precisely, the data received in step S70 relate to the health status of the patient measured after execution of the at least one recommendation 33.

The method may further comprise a step S80 of modifying the index risks 31 based on the information resulting from the user executing the at least one recommendation 33.

After the modification step S80, a calculation step S90 may be implemented to modify the global risk level 32 based on the modified index risks 31.

In one embodiment, the method further comprises repeating the steps S10 to S90 for a plurality of patients and generating a medical database comprising, for each patient of the plurality: the patient data 20; the index risk 31; the global risk level 32; the selected recommendations 33 and the information resulting from the user executing the at least one recommendation 33.

In one embodiment, one or more steps of the present method are undertaken by a perianesthetic assessment algorithm executed by the processing unit 3.

Preferably, said perianesthetic assessment algorithm is a rule-based algorithm.

The method may further comprise periodically analyzing data in a reference medical database comprising patient data 20 and patient outcomes, so as to identify correlations between at least one of the patient data 1 and a patient outcome.

More precisely, in this case the method may comprise receiving S100 data from said medical database and calculating S110 correlations between the received patient data 20 and the respective outcomes.

The method may further comprise periodically, interrogating a medical guideline database about updated guidelines.

In this case the method may comprise a step of receiving S120 medical guidelines, followed by a step of comparing S130 the received medical guidelines with prior medical guidelines, i.e., medical guidelines received at a preceding time.

More precisely, the receiving step S120 may comprise periodically receiving medical guidelines from at least one medical guideline database, each medical guideline being received at a respective time, the medical guidelines being stored in a database and being associated with a label (such as for instance “stroke prevention”).

Then, in the comparing step S130 each received medical guideline may be compared with a respective medical guideline, i.e. with a medical guideline associated with the same label and being received at a preceding time.

The method may further comprise a step of outputting S150 the modified first algorithm. Advantageously, the modified algorithm may be outputted so as to receive a user validation of the modification.

This embodiment allows to modify S140 or suggest a modification of the first algorithm based on the result of the analysis or the interrogation.

In one embodiment, the modified perianesthetic assessment algorithm is validated against a set of consistency rules and on a set of clinical reference cases.

The present invention also relates to a computer program product for assessing the risks of a patient undergoing an anesthesia procedure, the computer program product comprising instructions which, when the program is executed by a computer, cause the computer to carry out the steps of the method according to any one of the embodiments described hereabove.

The computer program product to perform the method as described above may be written as computer programs, code segments, instructions or any combination thereof, for individually or collectively instructing or configuring the processor or computer to operate as a machine or special-purpose computer to perform the operations performed by hardware components. In one example, the computer program product includes machine code that is directly executed by a processor or a computer, such as machine code produced by a compiler. In another example, the computer program product includes higher-level code that is executed by a processor or a computer using an interpreter. Programmers of ordinary skill in the art can readily write the instructions or software based on the block diagrams and the flow charts illustrated in the drawings and the corresponding descriptions in the specification, which disclose algorithms for performing the operations of the method as described above.

The present invention further comprises a computer-readable storage medium comprising instructions which, when the program is executed by a computer, cause the computer to carry out the steps of the method according to any one of the embodiments described hereabove.

According to one embodiment, the computer-readable storage medium is a non-transitory computer-readable storage medium.

Computer programs implementing the method of the present embodiments can commonly be distributed to users on a distribution computer-readable storage medium such as, but not limited to, an SD card, an external storage device, a microchip, a flash memory device, a portable hard drive and software websites. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+ Rs, CD-RWs, CD+ RWs, DVD-ROMs, DVD-Rs, DVD+ Rs, DVD-RWs, DVD+ RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

### EXAMPLES

The present invention is further illustrated by the following non-limitative examples.

### Example 1: Mortality Prediction

### Materials and Methods

In this example, a prediction models for in-hospital mortality was established by using a machine learning algorithm on the basis of the global risk level. This study was performed on a study population comprising 3304 patients. Data collected from the patients were allocated to training dataset (n=3068) and test dataset (n=236).

The global risk level 32 was calculated for each patient by using the method compliant with the present disclosure, and the prediction model for mortality was established by using a machine learning algorithm on the basis of the global risk level.

The receiver operating characteristic (ROC) curve was computed to evaluate the performance of the model for mortality prediction with respect to observed in-hospital mortality.

### Results

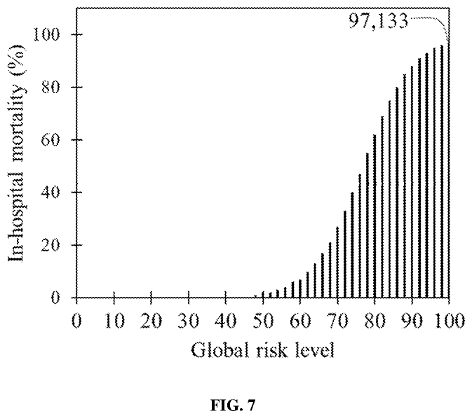

FIG. 7 illustrates the in-hospital mortality plotted against the global risk level.

The area under the ROC curve of the prediction model for mortality was 0.89.

These results respectively show the existence of a relationship between the global risk level and the in-hospital mortality, and that the model accurately predicted patient mortality on the basis of the global risk level.

### Example 2: Case Study

### Materials and Methods

In this example, the recommendations selected by a physician in absence of interaction with the system 1 of the present disclosure, and the recommendations selected by the system 1 were compared.

The recommendations were selected by the system 1 on the basis of the on the basis of the index risks and the global risk level.

The recommendations were selected for a woman undergoing hysterectomy.

### Results

The recommendations selected by the physician and the recommendations generated by the system, on the basis of the global risk level, differed in that the latter further comprised a preoperative iron injection.

The physician rated the recommendations selected by the algorithm as clinically relevant.

These results show that the system 1 is capable of providing recommendations which minimizes the index risks (in this case, the anemia risk), thereby providing a safer approach.

1-15. (canceled)

16. A computer-implemented method for assessing risks of a patient undergoing an anesthesia procedure, the method comprising the following steps:
receiving data relating to the patient health status, the received data comprising:
the patient's answers a questionnaire relating to at least a respiratory status and a cardiac status of the patient;
data collected from at least one sensor configured to sense physiological signals representative of the respiratory status and the cardiac status of the patient; and
data from a repository comprising an anesthetic information and management record and/or an electronic medical record of the patient;
labelling each of the received patient data according to a set of tags comprising a “respiratory” tag and a “cardiac” tag;
from the received patient data, calculating a number N of index risks, the index risks comprising at least one respiratory risk calculated based on the patient data labeled as “respiratory” and at least one cardiac risk calculated based on the patient data labeled as “cardiac”;
calculating a global risk level based on the index risks; and
outputting the global risk level so as to provide the risk of the patient undergoing an anesthesia procedure;
wherein the global risk level (32) is calculated via equation e1:
Σᵢ₌₁^{N}kᵢ*Rᵢ (e1),
N being the number of calculated index risks, k being a weighting factor, R being a numerical value associated with each calculated index risk.

17. The method according to claim 16, further comprising the following steps:
selecting at least one recommendation from a library of recommendations, the at least one recommendation being selected based at least on the global risk level;
receiving as input data relating to the health status of the patient measured after execution of the at least one recommendation;
modifying each of the N index risks based on the data relating to the health status of the patient measured after execution of the at least one recommendation; and
calculating an updated global risk level based on the modified index risks.

18. The method according to claim 16, further comprising the following steps:
periodically, receiving data from a medical database comprising patient data and respective outcomes;
calculating at least one correlation between the received patient data and the respective outcomes;
modifying the inputs of equation el based on the at least one correlation;
optionally, outputting the modified first algorithm.

19. The method according to claim 16, further comprising the following steps:
periodically, receiving medical guidelines from at least one medical guideline database, each medical guideline being received at a respective time, the medical guidelines being stored in a database and being associated with a label;
comparing each received medical guideline with the respective medical guideline associated with the same label and being received at a preceding acquisition time;
modifying the inputs of equation el based on the result of the comparison step;
optionally, outputting the modified first algorithm.

20. A computer program product for assessing the risks of a patient undergoing an anesthesia procedure, the computer program product comprising instructions which, when the program is executed by a computer, cause the computer to carry out the steps of the method according to claim 16.

21. A computer-readable storage medium comprising instructions which, when the program is executed by a computer, cause the computer to carry out the steps of the method according to claim 16.

22. A system for assessing risks of a patient undergoing an anesthesia procedure, the system comprising:
an input configured to receive data relating to the patient health status, the received data comprising:
the patient's answers to at least one questionnaire relating to at least a respiratory status and a cardiac status of the patient;
data collected from at least one sensor configured to sense physiological signals representative of the respiratory status and the cardiac status of the patient; and
data from a patient data repository;
a memory configured to store the patient data;
a processing unit configured to:
label each of the received data according to a set of tags comprising a “respiratory” tag and a “cardiac” tag;
based on the received data, calculate a number N of index risks, the index risks comprising at least one respiratory risk calculated based on the patient data labeled as “respiratory” and at least one cardiac risk calculated based on the patient data labeled as “cardiac”;
calculate a global risk level based on the index risks; and
an output for outputting the global risk level so as to provide the risk of the patient undergoing an anesthesia procedure,
wherein the global risk level is calculated via equation e1:
Σᵢ₌₁^{N}kᵢ*Rᵢ (e1),
N being the number of calculated index risks, k being a weighting factor, R being a numerical value associated with each calculated index risk.

23. The system according to claim 22, wherein the memory is further configured to store a library of recommendations, and the processing unit is further configured to:
select at least one recommendation from the library of recommendations based at least on the global risk level;
receive as input data relating to the health status of the patient measured after execution of the at least one recommendation;
modify each of the index risks based on the data relating to the health status of the patient measured after execution of the at least one recommendation;
calculating an updated global risk level based on the modified index risks.

24. The system according to claim 22, wherein the memory is further configured to store a reference medical database comprising patient data and patient outcomes collected from a plurality of patients and wherein the processing unit is further configured to:
periodically, receiving data from a medical database comprising patient data (20) and corresponding patient outcomes;
calculate at least one correlation between the patient data and patient outcomes;
modifying the inputs of the equation el based on the at least one correlation.

25. The system according to claim 22, wherein the processing unit is further configured to:
periodically, receiving medical guidelines from at least one medical guideline database, each medical guideline being received at a respective time, the medical guidelines being stored in a database and being associated with a label;
comparing each received medical guideline with the respective medical guideline associated with the same label and being received at a preceding time;
modifying inputs of the equation el based on the result of the comparison.

26. The system according to claim 22, wherein the processing unit is further configured to execute a machine learning algorithm configured to:
predict a patient outcome based on the patient data;
receive as input a measured patient outcome;
compare the predicted patient outcome with the received patient outcome to identify a discrepancy;
in case of discrepancy, anonymize and store the measured patient outcome and the patient data in the training dataset of the machine learning algorithm and/or in a reference medical database.

27. The system according to claim 26, wherein the patient predicted outcome and the measured outcome are selected from a group comprising:
future admissions or discharges;
per-anesthetic outcomes;
early or late post-anesthetic outcomes.

28. The system according to claim 22, wherein the at least one sensor is selected among: an optical sensor, a pressure sensor; a force sensor; a thermal sensor and the data collected from the at least one sensor comprise physiological data or identity data.

29. The system according to claim 22, wherein the output is further configured to:
output a first pre-operative anesthesia evaluation comprising the index risk, the global risk level and the at least one recommendation;
output a second pre-operative anesthesia evaluation comprising the selected at least one recommendation; information resulting from the user executing the at least one recommendation; the modified index risks and the updated global risk level.

30. The system according to claim 22, wherein the N index risks are selected from a group consisting of: respiratory risk, neurological risk, kidney failure risk, immunologic risk, chronic pain risk, vascular risk, hepatic risk, cardiac risk, airway management risk, nausea and vomiting risk, thromboembolic risk, hemorrhagic risk, allergy risk, anemia risk infection risk, and delirium risk.
